(19) United States
(12) Patent Application Publication
(10) Pub. No.: US 2018/0064434 A1
(43) Pub. Date: Mar. 8, 2018

(54) FLEXIBLE CONSTRUCTS

(71) Applicant: ARTHREX, INC., Naples, FL (US)

(72) Inventors: Jacob A. Jolly, Naples, FL (US); Arley Perez, III, Naples, FL (US); Anthony A. Laviano, Fort Meyers, FL (US); Andrew K. Osika, Naples, FL (US)

(21) Appl. No.: 15/255,286

(22) Filed: Sep. 2, 2016

(51) Int. Cl. A61B 17/04

(52) U.S. Cl. CPC A61B 17/0401; A61B 17/0487; A61B 2017/06176; A61B 2017/0445; A61B 2017/0464; A61B 2017/0412

(57) ABSTRACT

A flexible construct according to an exemplary aspect of the present disclosure includes, inter alia, a sheath and a suture connected to the sheath to form an adjustable loop. The suture includes a plurality of barbs that permit movement of the suture in a first direction relative to the sheath and restrict movement in a second direction relative to the sheath.

### BACKGROUND

This disclosure relates to surgical devices and methods for attaching body tissue, such as soft or hard tissue, to another piece of body tissue. More particularly, this disclosure is directed to flexible constructs that form loops using a barbed member to prevent the loop from loosening.

Orthopedic procedures are often performed to repair musculoskeletal injuries. For example, soft tissue may tear away from bone during vigorous exercise or sporting activities. When such tears occur, reattachment is often necessary to repair the damaged tissue. Suture anchors are one type of surgical device that has been developed to facilitate these repairs. Additional advancements in this field of technology are desired.

### SUMMARY

This disclosure relates to surgical fixation devices and methods. The surgical fixation devices include a flexible construct that forms an adjustable loop that can be tightened but restricts movement that would loosen the adjustable loop. The surgical fixation devices can be used in various tissue reconstruction procedures, including but not limited to, ACL and PCL reconstructions and capsular closure.

A flexible construct according to an exemplary aspect of the present disclosure includes, inter alia, a sheath and a suture connected to the sheath to form an adjustable loop. The suture includes a plurality of barbs that permit movement of the suture in a first direction relative to the sheath and restrict movement in a second direction relative to the sheath.

A surgical method according to an exemplary aspect of this disclosure includes, inter alia, attaching a first piece of tissue to a second piece of tissue using a tissue closure assembly. The tissue closure assembly includes a sheath and a barbed suture. The barbed suture includes a plurality of barbs that permit movement of the barbed suture in a first direction relative to the sheath and restrict movement in a second direction relative to the sheath.

A flexible construct according to another exemplary aspect of the present disclosure includes, inter alia, a suture forming an adjustable loop and a first splice in the suture. A barbed device is located at least partially within the splice and includes a first plurality of barbs directed in a first direction and a second plurality of barbs directed in a second direction.

A surgical method according to another exemplary aspect of this disclosure includes, inter alia, passing a suture through a first splice in a first direction and restricting movement of the suture through the first splice in a second direction with a barbed device.

### BRIEF DESCRIPTION OF THE DRAWINGS

FIG. 1 illustrates a flexible construct, such as a tissue closure assembly, according to one embodiment.Figure 2A illustrates a cross-sectional view of a sheath of the flexible construct of FIG. 1.

FIG. 2B schematically illustrates an embodiment for inserting a suture through a sheath of the flexible construct of FIG. 1.

FIG. 3A illustrates the flexible construct of FIG. 1 approximating tissue.

FIG. 3B illustrates a flexible construct according to another embodiment approximating tissue.

FIG. 4 illustrates a flexible construct according to another embodiment.

FIG. 5 illustrates a button used in connection with the flexible construct of FIG. 4.

FIG. 6 illustrates a cross-sectional view taken through splices in the flexible construct of FIG. 4.

FIG. 7 illustrates a flexible construct according to yet another embodiment.

FIG. 8 illustrates a cross-sectional view taken through a slice of the flexible construct of FIG. 7.

FIG. 9 illustrates a flexible construct according to a further embodiment.

### DETAILED DESCRIPTION

This disclosure relates to surgical fixation devices and methods. Surgical fixation devices include a flexible construct that forms an adjustable loop that can be tightened but restrict movement that would loosen the adjustable loop. The surgical fixation devices can be used in various tissue reconstruction procedures, including but not limited to, ACL and PCL reconstructions and capsular closure.

A tissue closure assembly according to an exemplary aspect of the present disclosure includes, inter alia, a sheath and a suture connected to the sheath to form an adjustable loop. The suture includes a plurality of barbs that permit movement of the suture in a first direction relative to the sheath and restrict movement in a second direction relative to the sheath.

In a further embodiment, a sheath includes a first sheath end and a second sheath end.

In a further embodiment, a suture includes a first suture end and a second suture end. A first suture end is attached to a first sheath end.

In a further embodiment, a plurality of barbs is directed towards a first suture end.

In a further embodiment, a first suture end is fixedly received within a sheath at a first sheath end.

In a further embodiment, a suture is fixedly attached to a sheath with an adhesive.

In a further embodiment, a second suture end is received in a second sheath end.

In a further embodiment, an exit hole is located in a sheath between a first sheath end and a second sheath end.

In a further embodiment, a second end of a suture is configured to be received within an exit hole.

In a further embodiment, a sheath is tubular in shape and is made of a fibrous material.

A surgical method according to an exemplary aspect of this disclosure includes, inter alia, attaching a first piece of tissue to a second piece of tissue using a tissue closure assembly. The tissue closure assembly includes a sheath and a barbed suture. The barbed suture includes a plurality of barbs that permit movement of the barbed suture in a first direction relative to the sheath and restrict movement in a second direction relative to the sheath.

In a further embodiment, a sheath includes a first sheath end and a second sheath end.

In a further embodiment, a barbed suture includes a first suture end and a second suture end. A first suture end is attached to a first sheath end.

In a further embodiment of the disclosed methods, an attaching step includes inserting a second suture end into the second sheath end and engaging a portion of a sheath with a plurality of barbs to prevent a second suture end from separating from the sheath.

In a further embodiment of the disclosed methods, an attaching step includes passing a second suture end through an exit hole in a side of a sheath.

In a further embodiment of the disclosed methods, an attaching step includes surrounding a first piece of tissue and a second piece of tissue with a barbed suture and a sheath.

In a further embodiment, a first piece of tissue and a second piece of tissue are portions of the same tissue.

A flexible construct according to another exemplary aspect of the present disclosure includes, inter alia, a suture and a first splice in the suture. A barbed device is located at least partially within the splice and is configured to reinforce the splice.

In a further embodiment, a first splice includes a first splice entrance and a first splice exit. A first portion of a barbed device is located between a first splice entrance and a first splice exit.

In a further embodiment, a barbed device includes a second portion located within a suture spaced from a first splice.

In a further embodiment, a first plurality of barbs and a second plurality of barbs are directed towards a mid-portion of a barbed device.

In a further embodiment, a first splice forms a first eyelet splice.

In a further embodiment, there are a second splice and a button. A second splice forms a second splice eyelet. The first eyelet splice and the second eyelet splice form an adjustable loop with the button.

A surgical method according to another exemplary aspect of this disclosure includes, inter alia, passing a suture through a first splice in a first direction and restricting movement of the suture through the first splice in a second direction with a barbed device.

In a further embodiment, a barbed device is located within an interior of the suture and includes a first plurality of barbs directed in a first direction and a second plurality of barbs directed in a second opposite direction.

In a further embodiment, a first plurality of barbs and a second plurality of barbs are directed towards a mid-portion of the barbed device.

In a further embodiment, a suture is passed through a second splice in a third direction and movement is restricted of the suture through the second splice in a fourth direction.

In a further embodiment, a suture forms an adjustable loop, and a barbed device prevents the adjust loop from expanding in size.

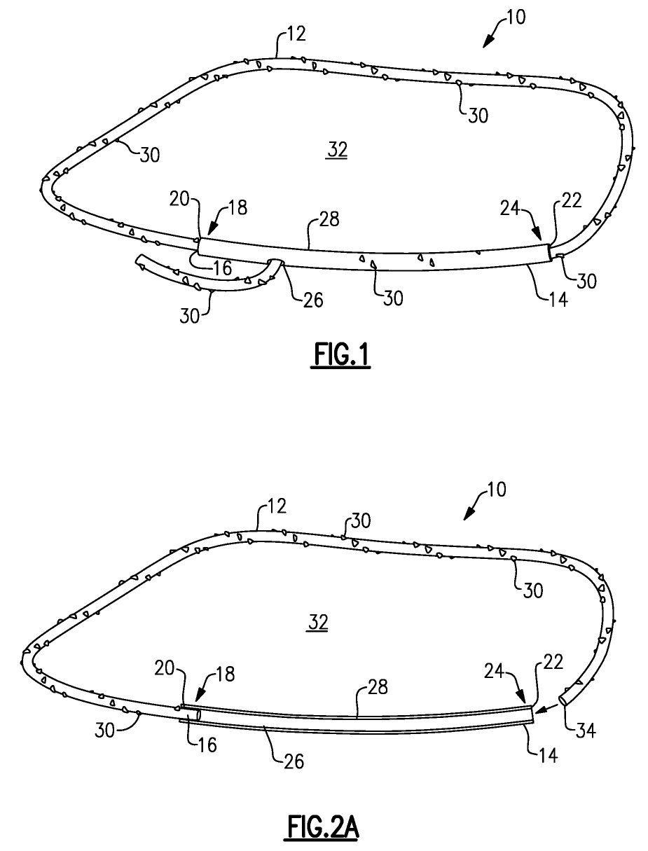

FIG. 1 illustrates a tissue closure assembly 10 according to one embodiment. The tissue closure assembly 10 includes a suture 12 and a sheath 14. In the illustrated embodiment, a first end 16 of the suture 12 is received within a first end portion 18 of the sheath 14. The sheath 14 includes an elongated tubular shaped wall 28 that defines a first opening 20 in the first end portion 18 and a second opening 22 in a second end portion 24 of the sheath 14. The sheath 14 may be made of a fibrous material, a polymer, or a composite material as non-limiting examples. An exit hole 26 is located between the first opening 20 and the second opening 22. The exit hole 26 extends through the tubular shaped wall 28 of the sheath 14.

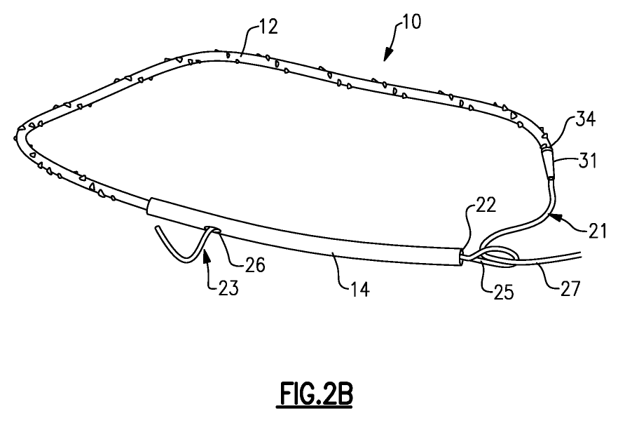

As shown in FIG. 2A, the first end 16 of the suture 12 includes an outer diameter that fits within an inner diameter of the first opening 20 of the sheath 14. The first end 16 of the suture 12 is attached to the sheath 14 with an adhesive or other bonding agent or process. The suture 12 includes multiple barbs 30 projecting outward an outer surface of the suture 12. The suture 12 may therefore be referred to as a barbed suture. In the illustrated embodiment, all of the barbs 30 are directed towards the first end 16 of the suture 12.

The suture 12 and the sheath 14 form a loop 32 when a second end 34 of the suture 12 is inserted into the second opening 22 in the sheath 14. Since the barbs 30 are oriented towards the first end 16 of the suture 12, the second end 34 of the suture 12 is able to move through the sheath 14 in a direction from the second opening 22 towards the first opening 20 but is prevented from moving in an opposite direction from the first opening 20 towards the second opening 22 by the barbs 30 engaging the sheath 14. A diameter of the loop 32 is reduced as the suture 12 is fed into the sheath 14. The loop 32 may be further tightened or constricted by pulling the second end 34 of the suture 12 until it has been inserted through the exit hole 26 in the sheath 14.

FIG. 2B illustrates an embodiment for inserting the suture 12 into the sheath 14 of the tissue closure assembly 10. The exemplary insertion method uses a leader suture 21 and a loading suture 23. The leader suture 21 may be attached to the second end 34 of the suture 12. In an embodiment, the leader suture 21 includes a cap 31 that is insertable over the second end 34 of the suture 12 to connect the leader suture 21 to the suture 12. The leader suture 21 aids in passing the suture 12 through tissue and subsequently through the sheath 14. The loading suture 23 is inserted through the exit hole 26 of the sheath and the second opening 22 of the sheath 14. The loading suture 23 may include a loop 25 configured to receive a tail 27 of the leader suture 21. The suture 12 can be shuttled into the sheath 14 by inserting the tail 27 through the loop 25 and then pulling the loading suture 23 through the sheath 14 by pulling the portion of the loading suture 23 that extends out of the exit hole 26.

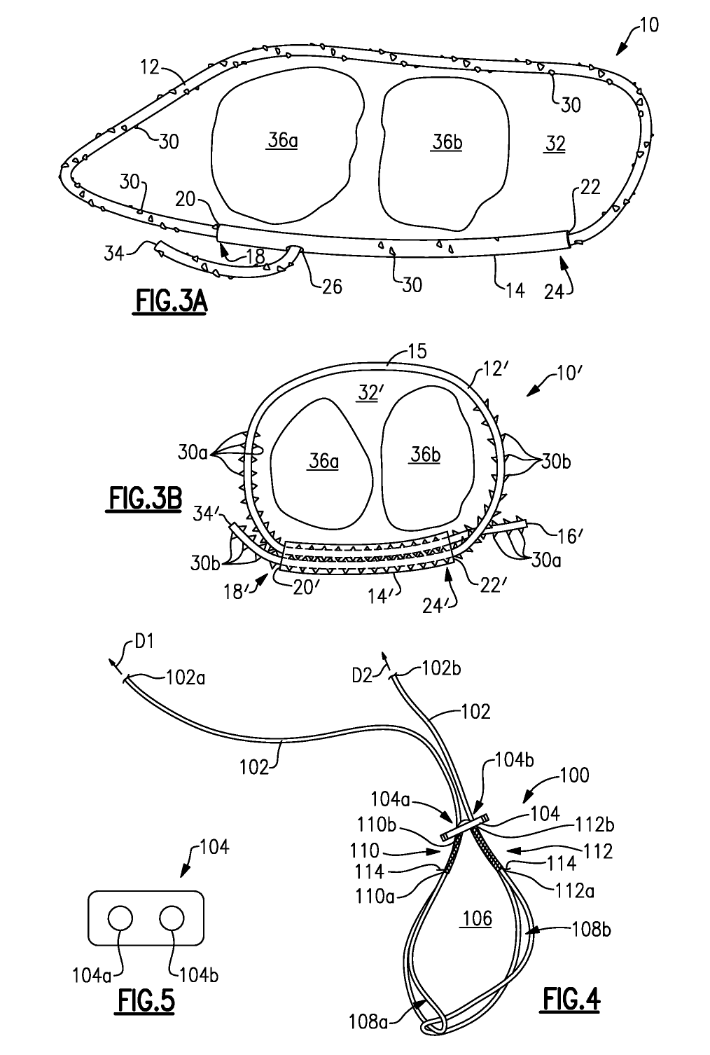

FIG. 3A illustrates an exemplary use of the tissue closure assembly 10. As shown in FIG. 3A, the tissue closure assembly 10 is used for approximating a first piece of tissue 36a relative to a second piece of tissue 36b. The first and second pieces of tissue 36a, 36b may be pieces of the same tissue or separate pieces of tissue. The tissue closure assembly 10 may also be used to approximate tissue in a capsular closure technique, for example.

The tissue closure assembly 10 of this disclosure provides an improved tissue closure device and technique. In particular, the barbed suture 12 reduces the complexity of forming a tissue securing loop by reducing the need to tie knots to form a loop which can be difficult and time consuming during arthroscopic procedures. Alternatively, the suture 12 and sheath 14 of the tissue closure assembly 10 could penetrate and pass through either the first and second pieces of tissue 36a, 36b.

FIG. 3B illustrates a tissue closure assembly 10′ according to another embodiment. The tissue closure assembly 10′ is similar to the tissue closure assembly 10 except where shown in the Figures or described below. The tissue closure assembly 10′ includes a suture 12′ and a sheath 14′. In the illustrated embodiment, the suture 12′ includes a first end 16′ and a second end 34.′ The sheath 14′ includes a first end portion 18′ having a first opening 20′ and a second end portion 24′ having a second opening 22′.

The suture 12′ includes a first plurality of barbs 30a and a second plurality of barbs 30b. The first plurality of barbs 30a is directed towards the second end 34′ or a mid-portion 15 of the suture 12′ and the second plurality of barbs 30b is directed towards the first end 16′ or the mid-portion 15 of the suture 12′.

In the illustrated embodiment, the first end 16′ of the suture 12′ is fed through the first opening 20′ in the sheath 14′ until the first end 16′ passes out of the second opening 22′ in the sheath 14′. Similarly, the second end 34′ of the suture 12′ is fed through the second end 22′ of the sheath 14′ until the second end 34′ passes out of the first opening 20′ of the sheath 14′. A size of a loop 32′ defined by the suture 12′ and the sheath 14′ can be reduced by pulling on the first and second ends 16′, 34′ of the suture 12′. The loop 32′ is prevented from expanding in size by the first plurality of barbs 30a engaging second plurality of barbs 30b. Moreover, the first and second plurality of barbs 30a, 30b engage an interior surface of the sheath 14′ to further prevent the loop 32′ from expanding.

The tissue closure assembly 10′ may also be used for approximating the first piece of tissue 36a relative to the second piece of tissue 36b, such as in a capsular closure technique, for example.

FIG. 4 illustrates a button/loop construct 100 that may be configured as a flexible construct according to another embodiment. The construct 100 includes a flexible strand of suture 102 and a button 104 that form an adjustable loop 106. In one example, the construct 100 can be used to connect tissue to bone for passing the button 104 through a bone tunnel in a first position generally parallel to the bone tunnel and then flipping the button 104 to a second position generally perpendicular to the bone tunnel. The adjustable loop 106 can be tightened by pulling a first end 102a and a second end 102b of the strand 102 in the directions D1 and D2, respectively. However, as will be described further below, the construct 100 includes a barbed device 114 that prevents the adjustable loop 106 from expanding and loosening once tightened by pulling the first and second ends 102a, 102b.

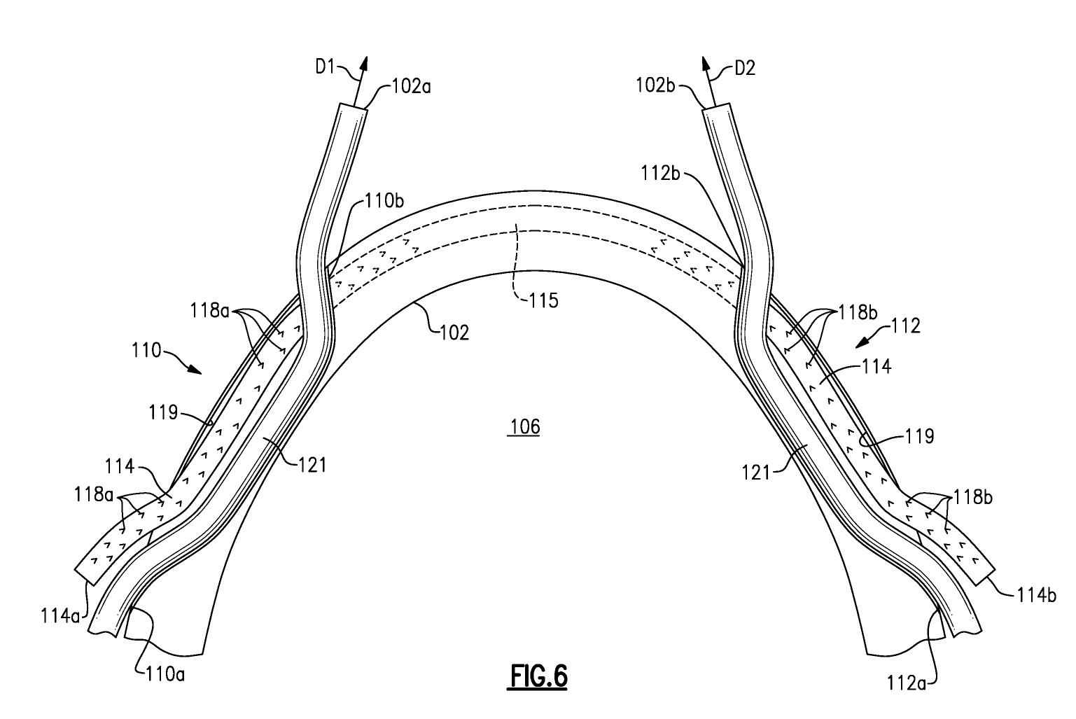

As shown in FIGS. 4 and 5, the construct 100 forms the adjustable loop 106 by threading the first end 102a of the strand 102 into a first opening 104a in the button 104 and then threading the second end 102b of the strand 102 through a second opening 104b in the button 104. Once the first and second ends 102a, 102b of the strand 102 have passed through the button 104, the first end 102a of the strand 102 is spliced back into the strand 102 at a first splice 110 to form a first eyelet splice 108a. The first end 102a may be spliced back into the strand with the assistance of a needle or other device. The first end 102a enters the first splice 110 at a first splice entrance 110a and exits the strand 102 at a first splice exit 110b. When the first end 102a exits the first splice exit 110b, the first end 102a is threaded back through the first opening 104a in the button 104. In the illustrated embodiment, the first splice exit 110b is located in close proximity to the first opening 104a in the button 104.

A second eyelet splice 108b is formed in a similar manner to the first eyelet splice 108a described above. After the second end 102b of the strand 102 is passed through the second opening 104b in the button 104, the second end 102b is passed through the first eyelet splice 108a to link the first eyelet splice 108a to the second eyelet splice 108b to form the adjustable loop 106. The second end 102b is then threaded into a second splice 112 in the strand 102. The second end 102b may be spliced back into the strand with the assistance of a needle or other device. The second end 102b enters the strand 102 at a second splice entrance 112a and exits the strand 102 at a second splice exit 112b. In the illustrated embodiment, the second splice exit 112b is located adjacent the second opening 104b in the button 104. The second splice exit 112b is space from the first splice exit 110b by a distance similar to the spacing between the first opening 104a and the second opening 104b in the button 104.

Once the first and second eyelet splices 108a, 108b have formed the adjustable loop 106, a size of the adjustable loop 106 can be reduced by pulling on the first and second ends 102a, 102b in the directions D1, D2, respectively. Once a desired size for the adjustable loop 106 is reached, the first end 102a of the strand 102 is fed through the second opening 104b in the button 104, and the second end 102b of the strand 102 is fed through the first opening 104a in the button 104. By feeding the first and second ends 102a, 102b through the button 104, the strand 102 is prevented from moving relative to the button 104 such that the size of the adjustable loop 106 will not increase.

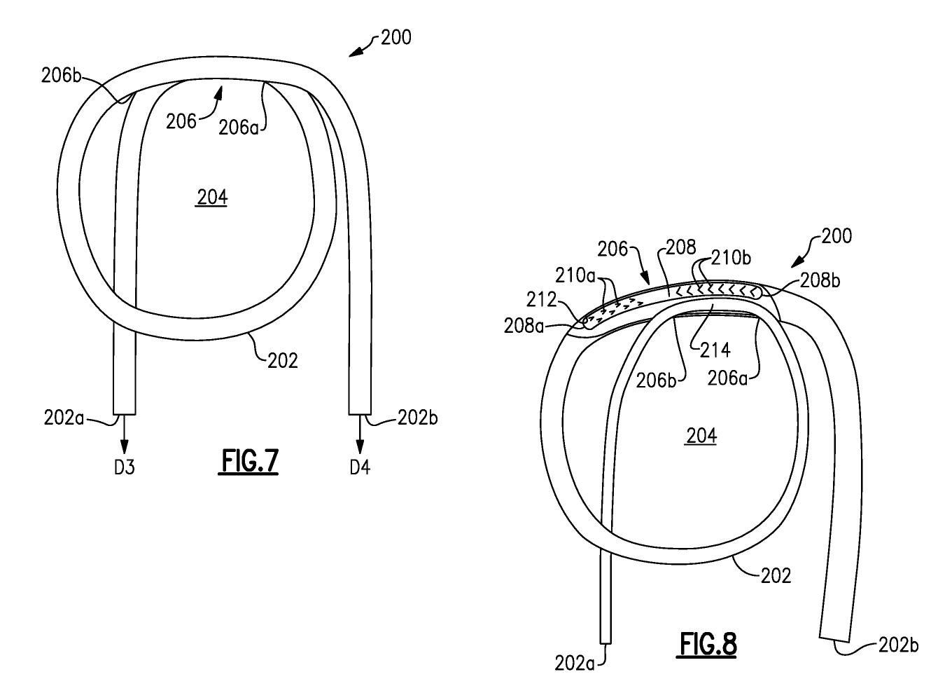

In addition to simply threading the first and second suture ends 102a, 102b back through the button 104, the construct 100 has additional fixation to secure the strand 102 from moving and increasing the size of the adjustable loop 106. This is accomplished with the use of the barbed device 114 as shown in FIG. 6.

FIG. 6 illustrates a cross-sectional view taken through the first and second splices 110, 112 of the construct 100 of FIG. 4 (shown without the button 104 for clarity). As shown in FIG. 6, the barbed device 114 extends between the first splice 110 and the second splice 112. In the illustrated embodiment, the barbed device 114 includes a first plurality of barbs 118a and a second plurality of barbs 118b. The first plurality of barbs 118a is located adjacent a first end 114a of the barbed device 114. The first plurality of barbs 118a is also at least partially located within an area of the first splice 110 and is oriented towards a mid-portion 115 of the barbed device 114. The second plurality of barbs 118b is located adjacent a second end 114b of the barbed device 114. The second plurality of barbs 118b is also at least partially located within an area of the second splice 112 and is oriented towards the mid-portion 115 of the barbed device 114. Since the first plurality of barbs 118a is oriented towards the mid-portion 115 of the barbed device 114, the strand 102 is able to pass through the first splice 110 when a force in the direction D1 is applied to the first end 102a. Similarly, because the second plurality of barbs 118b is oriented towards the mid-portion 115 of the barbed device 114, the strand 102 is able to pass through the second splice 112 when a force is applied in the direction D2.

Moreover, the barbed device 114 prevents the adjustable loop 106 from expanding and the first and second ends 102a, 102b from moving in a direction opposite D1, D2, respectively. In order to prevent expansion of the adjustable loop 106, the first plurality of barbs 118a engages an interior portion 119 of the strand 102 adjacent the first splice 110, and the second plurality of barbs 118b engages an exterior portion 121 of the strand 102 adjacent the second splice 112 when a force is applied to the adjustable loop 106 that would expand its size. Similarly, the second plurality of barbs 118b also engages the interior portion 119 of the strand 102 adjacent the second splice 112, and the first plurality of barbs 118a engages an exterior portion of the strand 102 adjacent the first splice 110 when a force is applied to the adjustable loop 106 that would expand its size.

In an embodiment, the barbed device 114 is a barbed suture. However, other devices may also be suitable for use as the barbed device 114 for reinforcing a spliced portion of a flexible construct.

FIG. 7 illustrates as a loop construct 200 a flexible construct according to yet another embodiment. The construct 200 includes a strand 202 that forms an adjustable loop 204. The adjustable loop 204 is formed by splicing the strand 202 at a first splice 206. The first splice 206 is formed by feeding a first end 202a of the strand 202 back through a portion of the strand 202 as shown in FIGS. 7 and 8.

The adjustable loop 204 can be adjusted by pulling on the first end 202a and a second end 202b of the strand 202 in the directions D3 and D4, respectively. However, as will be described further below, the construct 200 includes a barbed device 208 that prevents the adjustable loop 204 from expanding and loosening once tightened by pulling the first and second ends 202a, 202b.

As shown in the cross-sectional view of the first splice 206 of the construct 200 in FIG. 8, a portion of the strand 202 and the barbed device 208 are located adjacent the first splice 206. The barbed device 208 includes a first distal end 208a and a second distal end 208b. The first distal end 208a of the barbed device 208 is closer to the first end 202a of the strand 202, and the second distal end 208b of the barbed device 208 is closer to the second end 202b of the strand 202. The barbed device 208 includes a first plurality of barbs 210a and a second plurality of barbs 210b. The first plurality of barbs 210a is oriented in a direction towards the second end 208b of the barbed device 208, and the second plurality of barbs 210b is oriented towards the first end 208a of the barbed device 208.

When the construct 200 is tightened by pulling on the first and second ends 202a, 202b of the strand 202 in the directions D3, D4, respectively, the portion of the strand 202 within the first splice 206 is able to slide freely in a direction from a first splice entrance 206a to a first splice exit 206b. When a force is applied to the construct 200 that would expand the adjustable loop 204, the first plurality of barbs 208a engages an interior surface 212 of the of the strand 202, and the second plurality of barbs 208b engages an exterior surface 214 of the strand 202 adjacent the first splice 206.

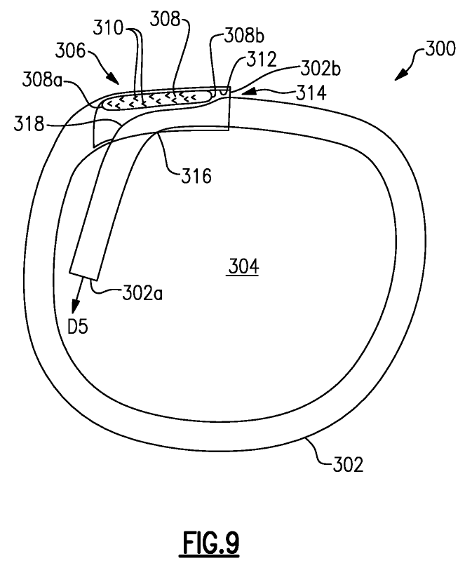

FIG. 9 illustrates as a loop construct 300 a flexible construct according to a further embodiment. The construct 300 includes a strand 302 that forms an adjustable loop 304. The adjustable loop 304 is formed by splicing the strand 302 at a first splice 306. The first splice 306 is formed by feeding a first end 302a of the strand 302 into an opening 314 defined by a second end 302b of the strand 302 and out a first splice exit 316.

The adjustable loop 304 can be adjusted by pulling on the first end 302a of the strand 302 in a direction D5. However, as will be described further below, the construct 300 includes a barbed device 308 that prevents the adjustable loop 304 from expanding and loosening once tightened by pulling the first end 302a.

As shown in the cross-sectional view of the first splice 306 of the construct 300 in FIG. 9, a portion of the strand 302 and the barbed device 308 are located adjacent the first splice 306. The barbed device 308 includes a first distal end 308a and a second distal end 308b. The first distal end 308a of the barbed device 308 is closer to the first end 302a of the strand 302 and the second distal end 308b of the barbed device 308 is closer to the second end 302b of the strand 302. The barbed device 308 includes a first plurality of barbs 310 oriented in a direction towards the first end 308a of the barbed device 308. The barbed device 308 may be secured to an interior of the strand 302 by an adhesive or other similar method.

When the construct 300 is tightened by pulling on the first ends 302a of the strand 302 in the directions D5, the portion of the strand 302 within the first splice 306 is able to slide freely in a direction from the opening 314 to the first splice exit 316. When a force is applied to the construct 300 that would expand the adjustable loop 304, the first plurality of barbs 310 engages an exterior surface 318 of the strand 302 adjacent the first splice 306.

The loop constructs 100, 200, and 300 of this disclosure provide an improved adjustable loop device and technique. The barbs on the barbed device prevent expansion of the adjustable loop once the loop has been tightened. This provides a stronger attachment with a reduction in the loop loosening after placement in the body.

In another embodiment, the barbed devices 114, 208, 308 of this disclosure can be utilized within any spliced construct. The barbed devices 114, 208, 308 can be positioned within the spliced portion of the construct for reinforcing a locking mechanism of the spliced portion.

Although the different non-limiting embodiments are illustrated as having specific components, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should also be understood that although a particular component arrangement is disclosed and illustrated in these non-limiting embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A tissue closure assembly, comprising:
a sheath; and
a suture connected to the sheath to form an adjustable loop, the suture having a plurality of barbs that permit movement of the suture in a first direction relative to the sheath and restrict movement in a second direction relative to the sheath.

2. The tissue closure assembly of claim 1, wherein the sheath includes a first sheath end and a second sheath end.

3. The tissue closure assembly of claim 2, wherein the suture includes a first suture end, a second suture end, and the first suture end is attached to the first sheath end.

4. The tissue closure assembly of claim 3, wherein the plurality of barbs is directed towards the first suture end.

5. The tissue closure assembly of claim 3, wherein the first suture end is fixedly received within the sheath at the first sheath end.

6. The tissue closure assembly of claim 5, wherein the suture is fixedly attached to the sheath with an adhesive.

7. The tissue closure assembly of claim 3, wherein the second suture end is received in the second sheath end.

8. The tissue closure assembly of claim 3 comprising an exit hole located in the sheath between the first sheath end and the second sheath end.

9. The tissue closure assembly of claim 8, wherein the second end of the suture is configured to be received within the exit hole.

10. The tissue closure assembly of claim 1, wherein the sheath is tubular in shape and is made of a fibrous material.

11. A method, comprising:
attaching a first piece of tissue to a second piece of tissue via a flexible construct including a sheath and a barbed suture having a plurality of barbs, wherein the plurality of barbs permits movement of the barbed suture in a first direction relative to the sheath and restrict movement in a second direction relative to the sheath.

12. The method of claim 11, wherein the sheath includes a first sheath end and a second sheath end.

13. The method of claim 12, wherein the barbed suture includes a first suture end and a second suture end, wherein the first suture end is attached to the first sheath end.

14. The method of claim 13, wherein said attaching step includes:
inserting the second suture end into the second sheath end; and
engaging a portion of the sheath with the plurality of barbs to prevent the second suture end from separating from the sheath.

15. The method of claim 14, wherein said attaching step includes:
passing the second suture end through an exit hole in a side of the sheath.

16. The method of claim 11, wherein said attaching step includes:
surrounding the first piece of tissue and the second piece of tissue with the barbed suture and the sheath.

17. The method of claim 11, wherein the first piece of tissue and the second piece of tissue are portions of the same tissue.

18. A flexible construct, comprising:
a suture;
at least one splice in the suture; and
a barbed device located at least partially within the splice and configured to reinforce the splice.

19. The flexible construct of claim 18, wherein the at least one splice includes a splice entrance and a splice exit; and a portion of the barbed device is located between the splice entrance and the splice exit.

20. The flexible construct of claim 19, wherein the barbed device includes a second portion located within the suture spaced from the at least one splice.

21. The flexible construct of claim 18, wherein the barbed device includes a first plurality of barbs and a second plurality of barbs directed towards a mid-portion of the barbed device.

22. The flexible construct of claim 18, wherein the at least one splice forms an eyelet splice.

23. The flexible construct of claim 22, wherein the at least one splice comprises a second splice and a button wherein the second splice forms a second eyelet splice and the first eyelet splice and the second eyelet splice form an adjustable loop with the button.

24. A method, comprising:
passing a suture through a first splice in a first direction; and
restricting movement of the suture through the first splice in a second direction with a barbed device.

25. The method of claim 24, wherein the barbed device is located within an interior of the suture and includes a first plurality of barbs directed in a first direction and a second plurality of barbs directed in a second opposite direction.

26. The method of claim 25, wherein the first plurality of barbs and the second plurality of barbs are directed in are directed towards a mid-portion of the barbed device.

27. The method of claim 25, including passing the suture through a second splice in a third direction and restricting movement of the suture through the second splice in a fourth direction.

28. The method of claim 24, wherein the suture forms an adjustable loop and the barbed device prevents the adjust loop from expanding in size.
